(19) United States
(12) Patent Application Publication
(10) Pub. No.: US 2025/0215328 A1
(43) Pub. Date: Jul. 3, 2025

(54) PROCESS FOR CATALYTIC CONVERSION OF PLASTICS WASTE INTO OLEFINS

(71) Applicant: Basell Poliolefine Italia S.R.L., Milano (IT)

(72) Inventor: Shahram Mihan, Bad Soden (DE)

(73) Assignee: Basell Poliolefine Italia S.R.L., Milano (IT)

(21) Appl. No.: 18/851,511

(22) PCT Filed: Mar. 30, 2023
(86) PCT No.: PCT/EP2023/058372

§ 371 Date: Sep. 26, 2024

(30) Foreign Application Priority Data
Mar. 30, 2022 (EP) 22165609.3

(51) Int. Cl. C10G 1/10

(52) U.S. Cl. CPC C10G 1/10; C10G 2300/1003; C10G 2300/70; C10G 2400/20

(57) ABSTRACT

A process for converting polymeric waste material into olefins via a depolymerization reaction. A process for the conversion of plastic waste into olefin, comprising: (i) depolymerizing a plastic waste feedstock comprising: (a) more than 80% wt of polyolefins and (b) less than 7.0 wt. % of non-polyolefin organic components, based on the total weight of the dry weight polymeric fraction of the waste material feedstock, thereby generating a gaseous fraction; (ii) collecting the gaseous fraction generated; and (iii) separating the collected gaseous fraction, thereby obtaining a gaseous and a liquid depolymerization product comprising higher than 60% wt of gaseous depolymerization product, based on total polyolefin content, wherein the gaseous depolymerization product comprised equal to or higher than 65% wt of C₂-C₄ olefins, based on the total amount of hydrocarbons.

### FIELD OF THE DISCLOSURE

In general, the present disclosure relates to the field of chemistry. More specifically, the present disclosure relates to polymer chemistry. In particular, the present disclosure relates to the depolymerization of polymeric waste materials.

### BACKGROUND OF THE DISCLOSURE

Plastics include a wide range of synthetic and semi-synthetic materials that use polymers as their main ingredient. Their plasticity makes it possible for plastics to be molded, extruded or pressed into solid objects of various shapes. This adaptability, in combination with a wide range of other properties, such as light weight, durability and low production cost, has led to their widespread use. The production of plastics has increased dramatically over the last few decades. At the same time, the increasing amount of plastics has given rise to environmental concerns. In some instances, plastics are resistant to natural degradation processes. As such, the material may persist for centuries or longer, filling up landfill sites and even appearing in the food chain as microplastics.

Therefore, efforts are undertaken to improve the recycling of polymeric waste materials. In some instances, procedures of recycling rely on mechanical recycling and chemical recycling. For mechanical recycling, the plastics are mechanically transformed without changing their chemical structure, thereby permitting the plastics to be used to produce new materials. In some instances, mechanical recycling steps include collecting polymeric waste material, sorting the polymeric waste material into different types of plastic and colors, packaging the plastic waste material by pressing or milling, washing the polymeric waste material, drying the polymeric waste material and reprocessing the polymeric waste material into pellets by agglutinating, extruding and cooling the plastic, thereby obtaining recycled raw material for use in new articles.

During chemical recycling, the plastics are reprocessed. In some instances, the structure and chemical nature of the plastics are modified, such that the plastics are usable as raw materials for different industries or as basic input or feedstock for manufacturing new polymeric products. In some instances, chemical recycling includes the steps of collecting plastic waste material and heating the plastic waste material, thereby yielding smaller organic molecules from breaking down the polymers. In some instances, the smaller organic molecules are recirculated in the petrochemical industry.

In some instances, the main effluent from the pyrolysis step is a liquid stream, also called pyrolytic oil. In some instances, the pyrolytic oil is refined and used as a fuel. In some instances, the pyrolytic oil is subjected to a further steam cracking step, thereby generating a gaseous fraction made from or containing C₂-C₄ olefins.

### SUMMARY OF THE DISCLOSURE

In a general embodiment, the present disclosure provides a process for the conversion of plastic waste into olefin, including the steps of:
(i) depolymerizing, at a temperature ranging from 400 to 700° C., in the presence of a catalyst, a plastic waste feedstock made from or containing
   (a) more than 80% wt of polyolefins and
   (b) less than 7.0 wt. % of non-polyolefin organic components,
   based on the total weight of the dry weight polymeric fraction of the waste material feedstock, thereby generating a gaseous fraction;
(ii) collecting the gaseous fraction; and
(iii) separating the collected gaseous fraction, thereby obtaining a gaseous and a liquid depolymerization product made from or containing higher than 60% wt of gaseous depolymerization product, based on the total polyolefin content, wherein the gaseous depolymerization product is made from or containing equal to or higher than 65% wt of C₂-C₄ olefins, based on the total amount of hydrocarbons.

In some embodiments, the plastic waste feedstock is made from or containing
(a) more than 80% wt of polyolefins and
(b) less than 7.0 wt. %, alternatively less than 6.0 wt. %, alternatively less than 5.0% wt, of
non-polyolefin organic components, based on the total weight of the dry weight polymeric fraction of the waste material feedstock.

### DETAILED DESCRIPTION OF THE DISCLOSURE

In some embodiments, the plastic waste feedstock is made from or containing more than 85 wt. %, alternatively more than 90 wt. %, alternatively more than 95 wt. %, of polyolefin content, based on the total weight of the polymeric waste material feedstock. In some embodiments, the polyolefin is selected from the group consisting of polypropylene (PP) and polyethylene (PE).

In some embodiments, the upper limit of polyolefin content is 99 wt %, alternatively 98 wt %, alternatively 97 wt %, based on the total amount of plastic waste feedstock.

In some embodiments, the weight ratio PE/PP in the polymeric waste material feedstock is equal to or higher than 2, alternatively equal to or higher than 3.5, alternatively equal to or higher than 5, alternatively equal to or higher than 6.

In some embodiments, the plastic waste feedstock is further made from or containing less than 35 wt. %, alternatively less than 30 wt. %, of total ash, determined as residue after heating the polymeric waste material feedstock at 800° C. for 120 hours in air.

In some embodiments, the plastic waste feedstock (i) is in shredded form and has a bulk density from 70 to 500 g/l, alternatively from 100 to 450 g/1, or (ii) is in pellet form and has a bulk density from 300 to 700 g/l, the bulk density being determined according to DIN 53466. It is believed that the specified bulk density ranges (a) facilitate a continuous depolymerization process, (b) prevent blockage of feeding lines and reactor fouling, (c) helps obtain low amounts of residues, and (d) increases the product yield. In some embodiments, the polymeric waste material is obtained from a shredded pipe with a particle size of <50 mm, alternatively <30 mm, alternatively <20 mm, alternatively <15 mm.

In some embodiments, the plastic waste feedstock is further made from or containing (i) less than 4%, alternatively less than 3%, of total volatiles (TV), measured as the weight loss of a 10 g sample at 100° C. after 2 hours at 200 mbar.

In some embodiments, the plastic waste material feedstock is further made from or containing other polymeric materials, alternatively materials formed from synthetic polymers. In some embodiments, the other polymeric materials include polyolefins other than PE and PP, polystyrene, polyethylene terephthalate (PET), polyvinyl chloride (PVC), polyamide, polycarbonate, polyurethane, polyester, natural and synthetic rubber, tires, filled polymers, composites and plastic alloys, and plastics dissolved in a solvent. In some embodiments, the other polyolefins are selected from the group consisting of polybutene-1 and ethylene-propylene elastomers.

In some embodiments and to maximize production of light gas olefins, the plastic feedstock is made from or containing polyolefins and less than 7.0 wt. %, alternatively less than 6.0 wt. %, alternatively less than 5.0% wt, of non-polyolefin polymeric materials, based on the total amount of plastic feedstock.

In some embodiments, the plastic waste material feedstock is made from or containing a single type of polyolefin waste material. In some embodiments, the plastic waste material feedstock is made from or containing a mixture of two or more different polymeric waste materials. In some embodiments, the polyolefin waste material is polyethylene (PE).

In some embodiments, the polyolefin waste material is crosslinked polyethylene. In some embodiments, the crosslinked polyethylene is selected from the group consisting of peroxide-crosslinked polyethylene, silane-crosslinked polyethylene, irradiation-crosslinked polyethylene, radically crosslinked polyethylene, azo-crosslinked polyethylene, UV initiated radically crosslinked polyethylene, and mixtures thereof.

In some embodiments, the plastic waste material feedstock is provided in different forms. In some embodiments and in small scale operations, the polymeric waste material feedstock is in the form of a powder. In some embodiments and in large scale operations, the polymeric waste material feedstock is in the form of pellets or in the form of shredded flakes or small pieces of film. In some embodiments, the pellets have a particle size from 1 to 20 mm, alternatively from 2 to 10 mm, alternatively from 2 to 8 mm. In some embodiments, the shredded flakes or small pieces of film have a particle size from 1 to 20 mm. As used herein, a particles size in a defined range means that 90 wt. % of the particles have a particle size which is in the defined range. In some embodiments, particle size is determined by sieving or by using a Beckman Coulters LS13320 laser diffraction particle size analyzer.

In some embodiments, a plastic waste material is made from or containing mostly plastic material. In some embodiments, a plastic waste material is named after the type of polymer which forms the predominant component of the polymeric waste material. In some embodiments, the plastic waste material employed as feedstock is made from or containing more than 50 wt. %, alternatively more than 60 wt. %, alternatively more than 70 wt. %, of the polymeric material, based upon the total weight of the plastic waste material. In some embodiments, the polymeric waste material feedstock is further made from or containing additives. In some embodiments, the additives are selected from the group consisting of fillers, reinforcing materials, processing aids, plasticizers, pigments, light stabilizers, lubricants, impact modifiers, antistatic agents, inks, and antioxidants.

In some embodiments, the polymeric waste materials are made from or containing polyolefins. In some embodiments, the polyolefins are selected from the group consisting of high-density polyethylene (HDPE), linear low-density polyethylene (LLDPE), low-density polyethylene (LDPE), ethylene-propylene-diene monomer (EPDM), and polypropylene (PP). In some embodiments, the polymeric waste materials are made from or containing a mixture of polyolefins with less than 3%, alternatively less than 2% wt, of polystyrene (PS).

In some embodiments, the polymeric waste material is free of other non-polyolefin polymeric waste materials. In some embodiments, the polymeric waste material is further made from or containing less than 3%, alternatively less than 2%, of other non-polyolefin polymeric waste materials, based on the total weight of the dry weight polymeric waste material feedstock. In some embodiments, the other non-polyolefin polymeric waste materials are selected from the group consisting of polyamide, polyvinyl chloride, polyvinylidene chloride, polyethylene terephthalate, polyurethane (PU), acrylonitrile-butadiene-styrene (ABS), nylon, and fluorinated polymers.

In some embodiments, the polymeric waste material is free of thermosetting polymers. In some embodiments, the polymeric waste material is further made from or containing less than 3 wt. %, alternatively less than 2 wt. %, of thermosetting polymers, based upon the total weight of the polymeric waste material feedstock.

In some embodiments, the plastic waste materials are selected from the group consisting of single plastic waste, mixed plastics waste, and rubber waste. In some embodiments, the plastic waste materials are selected from the group consisting of single plastic waste, single virgin plastic off spec, mixed plastics waste, rubber waste, and mixtures thereof. In some embodiments, the plastic waste materials are selected from the group consisting of single virgin plastic off-spec, mixed plastics waste, and mixtures thereof.

In some embodiments, the plastic waste material is further made from or containing limited quantities of non-pyrolysable inorganic components. In some embodiments, the non-pyrolysable inorganic components are selected from the group consisting of water, glass, stone, and metal. In some embodiments, the non-pyrolysable inorganic components are contaminants. As used herein, the term “limited quantities” refers to an amount of less than 35 wt. %, alternatively less than 30 wt. %, of the total weight of the dry plastic waste material feedstock.

In some embodiments, the plastic waste material is extruded prior to being employed as feedstock in the process. In some embodiments, the plastic waste material is pelletized, wherein the pellets are employed as feedstock. In some embodiments, the plastic waste material is employed in a molten state. In some embodiments, the plastic waste material is in a molten state at temperatures from 200° C. to 300° C.

In some embodiments, the plastic waste material employed as feedstock:
i) has a polyolefin content of more than 80 wt. %, alternatively more than 85 wt. %, alternatively more than 90 wt. %, alternatively more than 95 wt. %, based on the total weight of the polymeric waste material feedstock;
ii) is shredded or in pellet form;
iii) has a total content of volatiles (TV) of less than 5%, alternatively less than 3%, alternatively less than 2%, alternatively less than 1%, measured as the weight loss of a 10 g sample at 100° C. and a pressure of 200 mbar after 2 hours;
iv) has an amount of polar polymer contaminants of less than 3 wt. %, alternatively less than 2 wt. %, based on the total weight of the polymeric waste material;
v) has an amount of cellulose, wood, or paper of less than 5 wt. %, alternatively less than 3%, based on the total weight of the polymeric waste material;
vi) has a total chlorine content of less than 1.0 wt. %, alternatively less than 0.5 wt. %, alternatively less than 0.1 wt. %, based on the total weight of the polymeric waste material; or
vii) has a total ash content of less than 10 wt. %, alternatively less than 5 wt. %, alternatively less than 3 wt. %, determined as residue after heating the polymeric waste at 800° C. for 120 hours in air. In some embodiments, the ash content is from 0.01 to 2 wt. %, alternatively from 0.02 to 1.5 wt. %, alternatively from 0.05 to 1.0 wt. %. In some embodiments, the polyolefin content is made from or containing polypropylene (PP), polyethylene (PE), or both. In some embodiments, the polymeric waste material is shredded, compacted, and has a bulk density from 70 to 500 g/l, alternatively from 100 to 450 g/l, determined according to DIN 53466. In some embodiments, the polymeric waste material is in pellet form and has a bulk density from 300 to 700 g/l, determined according to DIN 53466.

In some embodiments, the plastic waste materials are defined by upper limits of minor components, constituents or impurities expressed as percent by weight. In some embodiments, the lower limits for the amounts of these components, constituents or impurities are below the detection limit, alternatively are 0.001 wt. %, 0.01 wt. %, or 0.1 wt. %, respectively.

In some embodiments, the polymeric waste stream is separated using a technique selected from the group consisting of moving beds, drums and screens, and air separators, thereby differentiating materials by size, weight and density. In some embodiments, advanced sorting of plastic waste is achieved by spectroscopy techniques (MIR, NIR [near-infrared]), X-ray or fluorescence spectroscopy, thereby providing plastic waste streams with high polyolefin content.

In some embodiments, Automatic Separation Techniques of waste plastics include dry sorting technique, electrostatic sorting technique, mechanical sorting method (involves centrifugal force, specific gravity, elasticity, particle shape, selective shredding and mechanical properties), wet sorting technique, and chemical sorting methods. In some embodiments, the wet sorting technique is a sink float sorting method.

In some embodiments, the sorting techniques are described in B. Ruj et al: Sorting of plastic waste for effective recycling, Int. J. Appl. Sci. Eng. Res 4, 2015, 564-571.

### Depolymerization

In some embodiments, the process for the depolymerization of plastic waste material, includes the step of pyrolyzing the plastic waste material, optionally in the presence of a catalyst, at a temperature ranging from 400 to 700° C., thereby providing a gaseous effluent.

In some embodiments, the gaseous effluent from the pyrolysis reactor is then (a) collected and (b) separated into a gaseous and a liquid depolymerization product.

In some embodiments, the depolymerization reaction is carried out at temperatures ranging from 420 to 600° C., alternatively from 420 to 550° C.

In some embodiments, the collected gaseous fractions are separated into liquid and gaseous depolymerization products by condensation. In some embodiments, the liquid depolymerization product and the gaseous depolymerization product are further processed.

In some embodiments, the process generates little to no char. In some embodiments, the residue of the depolymerization process has a char content of less than 5 wt. %, alternatively less than 2 wt. %, based on the total weight of the depolymerization product.

In some embodiments, the liquid depolymerization product is further separated. In some embodiments, the process further includes a step of distilling the liquid depolymerization product.

In some embodiments, the process yields a depolymerization product with a high gaseous content. In some embodiments, the gaseous content in the depolymerization product is more than 70 wt. %, alternatively more than 75 wt. %, alternatively more than 80 wt. %, based on the weight of the polymeric fraction of the plastic waste feedstock.

In some embodiments, the gaseous fraction of the depolymerization product is made from or containing a high content of monomeric olefinic C₂-C₄-compounds. In some embodiments, the monomeric olefinic C2-C4-compounds are useful for further processing. In some embodiments, the monomeric olefinic C2-C4-compounds are for the production of polymers. In some embodiments, the amount of olefinic C₂-C₄-compounds is equal to or higher than 70%, alternatively equal to or higher than 72%, alternatively equal to or higher than 75%, based on the total amount of hydrocarbons in the gaseous fraction.

In some embodiments, the amount of C₂-C₄ hydrocarbons in the gaseous fraction of the depolymerization product is higher than 80%, alternatively higher than 85%, alternatively higher than 90%, based on the total amount of hydrocarbons in the gaseous fraction. In some embodiments and due to the high amount of such compounds generated during depolymerization, the gaseous depolymerization product is directly used as feedstock in cracking processes and subsequent polymerization. In some embodiments, the gaseous product is made from or containing light olefins and light alkanes. In some embodiments, the gaseous product transferred to a downstream cracker, bypassing the ovens and thereby producing polymerization grade monomer streams.

In some embodiments, other side products are cracked in the oven. In some embodiments, the other side products are selected from the group consisting of ethane, propane, and butane. In some embodiments, the steps of treating the depolymerization product to obtain the monomers are bypassed, thereby saving energy and reducing CO₂ output.

In some embodiments, the depolymerization process is carried out in a reactor including: (a) feeding devices for introducing polymeric waste material and catalyst into the reactor; (b) a pyrolysis device equipped with heating units, gas discharge units and a solid discharge unit; and (c) a condensation device.

In some embodiments, the gas discharge units are distributed throughout the pyrolysis device and are provided with an outlet to discharge the gaseous fraction of the depolymerization and an inlet for introducing cleaning gas into the pyrolysis device.

In some embodiments, the reactor includes more than a single pyrolysis unit.

In some embodiments, the polymeric waste feedstock and the catalyst are introduced into the pyrolysis unit via a feeding device and then heated, thereby achieving depolymerization and generating gaseous fractions. In some embodiments, the polymeric waste feedstock and the catalyst are introduced via more than a single feeding device. In some embodiments, the gaseous fractions are discharged through the outlet of the gas discharge units and conveyed to the condensation unit for further processing. In some embodiments, solid residue of the depolymerization is discharged via the solid discharge unit. In some embodiments, cleaning gas for cleaning the gas discharge units and the pyrolysis unit is introduced through the inlet of the gas discharge units.

In some embodiments, the gas discharge units are equipped with filter membrane, thereby avoiding the presence of solids in the gaseous fractions after being discharged from the pyrolysis device. In some embodiments, the gas discharge units are made from or containing metallic, ceramic grain, or fiber materials.

In some embodiments, the pyrolysis device is further equipped with a screw for homogenously mixing the polymeric waste material in the pyrolysis device throughout the depolymerization. In some embodiments, the residence time of the solids in the pyrolysis device is defined by adjusting the rotational speed of the screw. In some embodiments, the residence time of the waste material is no more than 60 minutes, alternatively no more than 45 minutes. In some embodiments, the pyrolysis device is operated at temperatures of 400 to 550° C.

In some embodiments, the discharged gaseous fractions of the depolymerization are conveyed to the condensation device, thereby obtaining a liquid and a gaseous depolymerization product. In some embodiments, the condensation device includes several condensers. In some embodiments, the condensers are operated at different temperatures. In some embodiments, the temperatures of the condensers are set according to the boiling points of the condensates.

In some embodiments, a catalyst is used in the depolymerization stage of the process.

In some embodiments, this catalyst is not a Fluid Catalytic Cracking catalyst.

In some embodiments, the catalyst has, as the active component, an acidic compound deposited on a particulate non-porous support with the aid of a coating agent.

In some embodiments, the catalyst facilitates producing pure depolymerization products with high content of gaseous fractions and low char generation. In some embodiments, the catalyst is separable from solid residue of the depolymerization process, thereby allowing for multiple uses of the catalyst.

In some embodiments, the catalyst is in particulate form. In some embodiments, particulate non-porous support is selected from the group consisting of sand, glass beads and metal particles. In some embodiments, the particulate non-porous support has any shape. In some embodiments, the shape is selected from the group consisting of spherical, cylindrical, and non-homogenous shapes. As used herein, the term “non-porous” refers to state of being not permeable to gases or liquids. In some embodiments, the gas is air. In some embodiments, the liquid is water. In some embodiments, the support has an average particle size D50 of 0.2 to 20 mm, alternatively 0.5 to 10 mm, alternatively 1 to 8 mm, alternatively from 1 to 6 mm, determined according to sieve analysis in accordance with ISO 3310-1/ASTM E11. In some embodiments, the test sieve apparatus of Retsch with woven wire mesh sieves (Ø 125 mm-20 μm) is used as the sieving device.

In some embodiments, sand is the non-porous particulate support. In some embodiments, the sand has a particle size distribution of:

| | Min | Max |
|---|---|---|
| <3000 μm | 70% | 90% |
| >1400 μm | 40% | 80% |
| >1000 μm | 50% | 80% |
| <1000 μm | 0% | 20% |

In some embodiments, the acidic compound of the catalyst is selected from the group consisting of Al/Si mixed oxides, Al₂O₃, aluminosilicates, silica and zeolites. As used herein, the term “Al/Si mixed oxides” refers to a material made from or containing a mixture of Al₂O₃ and SiO₂, having a neutral structure.

As used herein, the term “zeolites” refers to crystalline microporous aluminosilicates, which are built up from corner-sharing SiO₄₋ and AlO₄₋ tetrahedrons, having the structure Mₙ+x/n [AlO₂]-x(SiO₂)_{y}]+zH₂O with n being the charge of the cation M and z defining the number of water molecules incorporated into the crystal structure. In some embodiments, M is an alkaline, alkaline earth metal, or hydrogen ion. In some embodiments, the ion is selected from the group consisting of H+, Na+, Ca2+, K+, and Mg2+. As described herein, zeolites differ from mixed Al/Si oxides by the zeolites' defined pore structure and ionic character. In some embodiments, the zeolite employed as the acidic compound is selected from the group consisting of Zeolite Y, Zeolite Beta, Zeolite A, Zeolite X, Zeolite L, and mixtures thereof, alternatively Zeolite Y and Zeolite Beta. In some embodiments, zeolites have the metal ion M substituted by a hydrogen. In some embodiments, the zeolite-type components are selected from the group consisting of ZSM-5, ZSM-11, ZSM-22, ZSM-23, ZSM-35, ZSM-48, ZSM-50, TS-1, TS-2, SSZ-46, MCM-22, MCM-49, FU-9, PSH-3, ITQ-1, EU-1, NU-10, silicalite-1, silicalite-2, boralite-C, boralite-D, BCA, and mixtures thereof.

In some embodiments, the acidic compound is an Al/Si mixed oxide. In some embodiments, the composition of the Al/Si mixed oxide is adjusted. In some embodiments, the acidic compound is made from or containing Al₂O₃ in an amount from 20 to 99 wt. %, alternatively from 30 to 80 wt. %, alternatively from 40 to 70 wt. %, based on the total weight of the acidic compound. In some embodiments, the acidic compound is made from or containing SiO₂ in an amount from 1 to 80 wt. %, alternatively from 20 to 70 wt. %, alternatively from 30 to 60 wt. %, based on the total weight of the acidic compound.

In some embodiments, the acidic compound contains an excess of Al₂O₃ with respect to SiO₂. In some embodiments, the weight ratio of Al₂O₃ to SiO₂ in the acidic compound is from 99:1 to 30:70, alternatively from 9:1 to 3:2, alternatively from 4:1 to 3:2.

In some embodiments, the determination of the SiO₂ and Al₂O₃ content of the acidic compound is carried out by atomic emission spectroscopy using an inductively coupled plasma (ICP-AES).

In some embodiments, the coating agent employed in the catalyst is selected from the group consisting of oil, inorganic hydrogel, and combinations thereof. In some embodiments, the inorganic hydrogel is silica hydrogel. In some embodiments, the oil is aromatic-free white mineral oil. In some embodiments, the aromatic-free white mineral oil is based on iso-paraffins. In some embodiments, the oil has a kinematic viscosity at 20° C. of 140 to 180 mm²/s, alternatively 150 to 170 mm²/s. In some embodiments, the oil has a kinematic viscosity at 40° C. of 40 to 80 mm²/s, alternatively 50 to 70 mm²/s. In some embodiments, the oil has a kinematic viscosity at 100° C. of 5 to 15 mm²/s, alternatively 7 to 10 mm²/s. In some embodiments, the kinematic viscosity is determined according to ISO 3104.

In some embodiments, the amount of coating agent ranges from 1 to 300% alternatively from 2 to 150% wt, alternatively from 5 to 100% wt, alternatively 10-80% wt, based on amount of acidic compound. For hydrogels, the amount to be employed is referred to the dry weight.

In some embodiments, the catalyst is made from or containing 0.5 to 6 wt. %, alternatively 2 to 4 wt. %, of the active compound, based on the total weight of the catalyst.

In some embodiments, the catalyst is obtained by mixing the particulate non-porous support and the coating agent, thereby obtaining a mixture, and then adding the acidic compound in the form of a powder to the resulting mixture. In some embodiments, the mixture is heat treated, thereby obtaining the catalyst. In some embodiments, the heat treatment is carried out at a temperature of 100 to 600° C. In some embodiments, the particulate non-porous support is subjected to a drying step before being mixed with the coating agent.

In some embodiments, the catalyst is obtained by a process including the steps of: (a) mixing the particulate non-porous support and the coating agent, thereby yielding a mixture; and (b) adding the acidic compound in powder form to the mixture of step (a).

In some embodiments, the catalyst is made from or containing: (a) sand as a particulate non-porous support; (b) an Al/Si mixed oxide or a zeolite as an acidic compound; and (c) mineral oil or silica hydrogel as a coating agent.

### Depolymerization Product

In some embodiments, the gaseous fractions generated during pyrolysis are separated into liquid and gaseous depolymerization products. In some embodiments, the separation occurs by condensation.

In some embodiments, the process yields a depolymerization product having selectivity for the gaseous fraction.

### Liquid Depolymerization Product

In some embodiments, the resulting liquid depolymerization product has a low content of aromatic compounds, alternatively a low content of polycyclic aromatic compounds and asphaltanes. In some embodiments, the liquid depolymerization product has a low content of aromatic and olefinic components and a high degree of purity.

In some embodiments, the liquid depolymerization product has less than 10 mol %, alternatively less than 5 mol %, alternatively no more than 3 mol %, of aromatic components, measured as contents of aromatic protons in mol % as determined by 1H-NMR-spectroscopy.

In some embodiments, the liquid depolymerization product has a low content of olefinic compounds. In some embodiments, the liquid depolymerization product has less than 7 mol %, alternatively less than 5 mol %, alternatively less than 3 mol %, of olefinic compounds, determined based on the contents of olefinic protons, based on the total number of hydrocarbon protons, as determined by 1H-NMR-spectroscopy.

In some embodiments, the content of double bonds is measured by the Bromine number (BrNo.) which indicates the degree of unsaturation. In some embodiments, the liquid depolymerization product has a Bromine number, expressed as gram bromine per 100 grams of sample, of less than 150, alternatively from 10 to 100, alternatively from 15 to 80, alternatively from 20 to 70, alternatively from 25 to 100, determined according to ASTM D1159-01.

In some embodiments, the liquid depolymerization product has a boiling range from 30 to 650° C., alternatively from 50 to 250° C. In some embodiments, the depolymerization product is separated into hydrocarbon fractionations of different boiling ranges. In some embodiments, the hydrocarbon fractions are selected from the group consisting of a light naphtha fraction made from or containing C₅ and C₆ hydrocarbons having a boiling range from 30° C. and 130° C., a heavy naphtha fraction made from or containing C₆ to C₁₂ hydrocarbons having a boiling range from 130° C. to 220° C., a kerosene fraction made from or containing C₉ to C₁₇ hydrocarbons having a boiling range from 220° C. to 270° C., and other high boiling point fractions selected from the group consisting of diesel fuel, fuel oil, and hydrowax. In some embodiments, the separation occurs by distillation.

In some embodiments, the liquid depolymerization product has little to no solid residue. In some embodiments, the content of residues, upon evaporation of the liquid depolymerization product, is no more than 5 ppm (w), determined according to ASTM D381.

### Gaseous Depolymerization Product

In some embodiments, the gaseous depolymerization product has a low content of low molecular hydrocarbons such as methane or ethane. In some embodiments, the gaseous depolymerization product is made from or containing high amounts of higher olefins. In some embodiments, the higher olefins are selected from the group consisting of ethylene, propylene, and butene.

In some embodiments, the amount of olefinic C₂-C₄-compounds is equal to or higher than 70%, alternatively equal to or higher than 72%, alternatively equal to or higher than 75%, based on the total amount of hydrocarbons in the gaseous depolymerization product. In some embodiments, the gaseous depolymerization product is made from or containing a high content of olefins selected from the group consisting of ethylene, propylene, and butene. In some embodiments, the gaseous depolymerization product has a low content of saturated low molecular hydrocarbons, alternatively hydrocarbons of the formula CₙH₂ₙ₊₂ wherein n is a real number ranging from 1 to 4.

In some embodiments, the gaseous depolymerization product has a content of methane of at most 7 wt. %, alternatively at most 5 wt. %, alternatively at most 3 wt. %, alternatively at most 2 wt. %, alternatively at most 0.5-1.5 wt. %, based on the total weight of the gaseous depolymerization product.

In some embodiments, the gaseous fraction has a high olefinic content and is used directly as feedstock for further processing in a cracker downstream, thereby obtaining purified monomer streams, and thereafter for the subsequent production of polymers, thereby bypassing the energy-consuming stream cracking ovens and reducing the output of CO₂. In some embodiments, the downstream cracker is a raw gas compressor.

In some embodiments, the gaseous depolymerization product has small quantities of HCl, HCN, H₂S, H₂O, NH₃, or COS. In some embodiments, the quantities of HCl, HCN, H₂S, H₂O, NH₃, or COS are removed in a purification step before introduction to the steam cracker backend segment.

The present disclosure will be explained in more detail with reference to the examples provided below.

### EXAMPLES

The following analytical methods were employed:
1) GC MS was used for liquid and gas analysis.
2) Char residue was determined according to mass balance after decoking the residues of the reactor at 800° C.
3) Liquid contents were characterized using simulated distillation (SimDist) analysis according to ASTM D 7213:2012. Final boiling point (FBP), boiling temperature at 50% and initial boiling point (IBP) were taken from SimDist.
4) The total content of unsaturated components in the liquid condensates were characterized via Bromine number determination using an 848 Titrino Plus (Metrohm AG, Herisau, Switzerland), equipped with a double PT-wire electrode, which had integrated PT1000 temperature sensor, and a 10 ml burette, in accordance with ASTM D1159-01 as described in Metrohm Application Bulletin 177/5e, December 2018. The Bromine number (BrNo.) represented the amount of bromine in grams absorbed by 100 grams of a sample.
5) 1H-NMR analysis was conducted by dissolving a sample of the liquid condensate in CDCl3 and characterizing the sample using proton NMR spectroscopy. Aromatic, olefinic and aliphatic protons were assigned according to the chemical shifts summarized in Table 1:

TABLE 1
Integral Regions in 1H-NMR spectroscopy

| Peak Assignment | 1H Chemical Shift (ppm) |
|---|---|
| I₁ (Aromatic Protons) | 8.25-7.27 |
| CDCl₃ -Solvent | 7.26 |
| I₂ (Aromatic Protons) | 7.25-6.60 |
| I₃ (Olefinic Protons - Type 2 ) | 6.60-5.95 |
| I₄ (Olefinic Protons - Type 1 ) | 5.95-5.67 |
| I₅ (Olefinic Protons - Type 2 ) | 5.67-5.35 |
| I₆ (Olefinic Protons - Type 3 ) | 5.35-5.15 |
| I₇ (Olefinic Protons - Type 1 ) | 5.15-4.85 |
| I₈ (Olefinic Protons - Type 4 ) | 4.85-4.40 |
| I₉ . . . (Paraffinic Protons) | 4.40-0.25 |

The listed types of olefinic protons were assumed to correspond to the following structures:

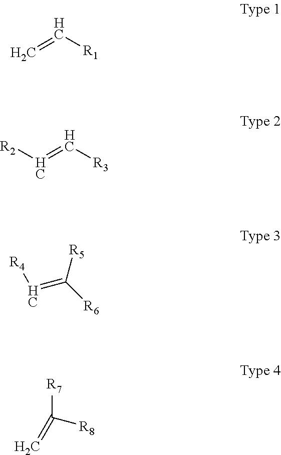

The amount of aromatic, olefinic and aliphatic protons were determined based on the assigned peak integrals according to the following equations:

$$Mol \% Aromatic Protons =$$

$$[ ( {I}_{1} + {I}_{2} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 1 =$$

$$[ ( {I}_{4} + {I}_{7} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 2 =$$

$$[ ( {I}_{3} + {I}_{5} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 3 =$$

$$[ ( {I}_{6} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 4 =$$

$$[ ( {I}_{8} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Paraffinic Protons =$$

$$[ ( {I}_{9} ) / ( {I}_{1} + {I}_{2} + {I}_{3} + {I}_{4} + {I}_{5} + {I}_{6} + {I}_{7} + {I}_{8} + {I}_{9} ) ] \%$$

6) The water content of the catalyst was determined using a Sartorius MA45 (Sartorius AG, Goettingen, Germany) on a sample of 0.5 to 1 g at 180° C.
7) For the determination of a pH value of the hydrodepolymerization products, a liquid sample of the hydrodepolymerization product was extracted with water in a volume ratio water:sample of 1:5. The pH value of the aqueous solution was measured.
8) Particle size distribution of the particulate non-porous support and the catalyst were determined according to Coulter counter analysis in accordance with ASTM D4438.
9) Properties of the organic waste material feedstock were determined as follows:

Samples of from 20 to 100 g of the polymeric waste were milled and analyzed. Alternatively, a pelletized sample of the polymeric waste was analyzed. The following methods were used:
i) Total Volatiles (TV) were measured as the weight loss of a 10 g sample at 100° C. and after 2 hours at 200 mbar.
ii) Water content was determined by Karl-Fischer titration, using an apparatus from Metrohm 915 KF Ti-Touch equipped with a PT100 indicator electrode for volumetric KF titration, according to Metrohm Application Bulletin 77/3e in compliance with ASTM E203.
iii) IR-Spectroscopy was used for a qualitative identification of various polymers (PP, PE, PS, PA, PET, PU, and Polyester) and additives such as CaCO₃.
iv) Standard elemental analysis was used for determination of wt. % of H, C, N (DIN 51732:2014-07) and S (tube furnace, ELTRA GmbH, Haan, Germany, DIN 51724-3:2012-07).
v) 1H-NMR was used for determining the composition of polymers soluble in solvents for recording a 1H-NMR spectrum: PE/PP balance (copolymers were also included), PET, PS.
vi) Ash Content analysis of plastics was determined at 800° C. according to DIN EN ISO 3451-1 (2019-05).
vii) Bulk density of the polymer waste was determined according to DIN 53466.
viii) Corrosivity was determined as the pH value of an aqueous solution after a contact time of 3 h (5 g sample in 50 ml distilled water).
ix) Inductively coupled plasma atomic emission spectroscopy (ICP-AES) was used for quantitative element determination (total chlorine content, content of Si or metals).
x) The ash content of a liquid feedstock such as pyrolysis oil, was measured according to ASTM D482-19.

In the catalyzed depolymerization runs, a catalyst supported on a non-porous support with the aid of a coating agent was used. Sand was used as the particulate non-porous support, having a particle size distribution, as summarized in Table 2, with 99% of the particles being smaller than 3 mm. Prior to use in formation of the catalyst, the sand was pre-dried at 80° C. for 24 h in a drying oven with circulating air.

TABLE 2
Particle size distribution

| | Sand, wt. % |
|---|---|
| >1400 μm | 40.55% |
| >1400 μm->1000 μm | 55.30% |
| <1000 μm | 4.15% |

### EXAMPLES

Catalysts using a mineral oil coating agent were prepared as follows:

25.0 kg of sand was placed into a 60 L steel barrel with screw cap and equipped with a Teflon inlay. 350 ml mineral oil were added (corresponding to 1.4 wt % with respect to sand). The drum was placed on a Drum Hoop Mixer and rotated for 1 hour (about 100 rpm). 24.5 kg of the resulting mixture was placed in another drum. 500 g of the acidic component (corresponding to a 2 wt % loading) were added. The drum was placed on a drum hoop mixer and rotated for 1 hour (about 100 rpm). At the end of the mixing process, a free-flowing catalyst was obtained with an even distribution of the particles of the acidic component on the surface of the sand particles.

The mineral oil used was Ondina X 432, which was commercially available from Shell, having the following properties:

TABLE 3

| Property | Method | Shell Ondina X 432 |
|---|---|---|
| Kinematic Viscosity at 20° C. in mm²/s | ISO 3104 | 165 |
| Kinematic Viscosity at 40° C. in mm²/s | ISO 3104 | 59 |
| Kinematic Viscosity at 100° C. in mm²/s | ISO 3104 | 9.0 |

Catalysts Using a Silica Hydrogel Coating Agent were Prepared as Follows:

25.0 kg of sand were placed into a 60 L steel barrel with screw cap and equipped with a Teflon inlay. 500 ml water were added (corresponding to 2.0 wt % with respect to sand). The drum was placed on a Drum Hoop Mixer and rotated for 1 hour (about 100 rpm). 24.5 kg of the resulting mixture was placed in another drum. 1000 g of a 1:1 milled free-flowing mixture of silica hydrogel and the acidic compound (corresponding to a 2 wt % loading) were added. The drum was placed on a drum hoop mixer and rotated for 1 hour (about 100 rpm). At the end of the mixing process, a free-flowing catalyst was obtained with an even distribution of the particles of the acidic compound on the surface of the sand particles. The resulting mixture was dried at 120° C. vacuum for 6 h.

The silica hydrogel was prepared according to the procedure described in European Patent Application No. EP1290042, example 1. The solid content of the hydrogel sample was 20 wt %. The D50 of the milled mixture of silica hydrogel and acidic components were between 80-100 μm in accordance with ASTM D4438.

Table 4 summarizes the catalysts employed with the amount of the acidic compound given in wt. % with respect to sand.

TABLE 4

| | | | Amount Acidic |
|---|---|---|---|
| Catalyst # | Coating Agent | Acidic Compound | Compound (wt. %) |
| 1 | silica hydrogel | Zeolyst Beta | 2 |
| 2 | oil | Zeolyst Beta | 2 |
| 3 | oil | ZSM-5 | 2 |

### Acidic Compounds:

Zeolyst Beta (CP811E-75), which was commercially available from PQ Corporation, Malvern, PA, USA

### Feedstock:

Some of the following organic waste materials were employed as feedstocks:
A: Pelletized agricultural and industrial packaging film.
   A1) Shredded Multilayer PEX pipe waste with an Aluminum layer (Particle size<20 mm)
B) Shredded PEX pipe waste with EVOH (Particle size<20 mm)
C) Shredded PEX pipe waste with an Aluminum layer (Particle size<20 mm)
D) Shredded monolayer PEX pipe waste (Particle size<20 mm)
E) Shredded high voltage cable crosslinked waste (Particle size<20 mm)
F) shredded and pelletized mixed plastic waste from household packaging (comparison)
G) Pelletized agricultural and industrial packaging film

The properties of the feedstocks, averaged on analysis of three samples, are summarized in Table 5.

TABLE 5

| | | | | | | Polymer composition analysis | | | | | |
|---|---|---|---|---|---|---|---|---|---|---|---|
| | | | | | | | | | | | Other |
| | Ash | TV | BD | Cl | PE + PP | PE | PP | PET | PS | PA | cont.* |
| Feedstock | wt % | wt % | g/cm³ | wt % | wt % | wt % | wt % | wt % | wt % | wt % | wt % |
| A | 1.5 | 0.1 | 356 | 0.01 | 95 | 83 | 12 | <0.1 | 1.7 | 0.7 | 2.6 |
| A1 | 25% | 0.5 | 383 | <0.01 | 75 | ~75 | — | — | — | — | 25 |
| B | 0.5 | 1 | 316 | <0.01 | | >95 | — | — | — | — | 2 |
| C | 11.2 | 1.5 | 326 | <0.01 | | >87 | — | — | — | — | 13 |
| D | <0.1 | 1.7 | 360 | <0.01 | | >98 | — | — | — | — | — |
| E | 6.5 | 0.6 | 480 | <0.01 | | >93 | — | — | — | — | 7 |
| F | 4.8 | 4 | 230 | 0.2 | 88 | 72 | 16 | 2.3 | 1.2 | — | 8.5 |
| G | 2.8 | 1 | 330 | 0.2 | 92 | 78 | 14 | — | — | — | 8 |

*Other content includes inorganic, polymer or organic contaminants and volatile components

Ash: ash content
TV: total volatiles
BD: bulk density
Cl: total chlorine content
PE: polyethylene content
PP: polypropylene content
PET: content of polyethylene terephthalate
PS: polystyrene content
PA: polyamide content
Other cont.: content of other contaminants

The feedstock and catalyst were introduced into a reactor device equipped with heating units, gas discharge units, solid discharge unit; a condensation device; and a screw for homogenously mixing the reactor content during depolymerization. Conditions of the depolymerization conducted are summarized in Table 6. The resulting gaseous fractions were further separated into liquid and gaseous depolymerization products by condensation. The amounts of the resulting fractions are also given in Table 6.

TABLE 6
Process parameter and mass balance ^{(a)}

| Run# | Temp | Time | Cat# | Feedstock | % Wax | % Liquid⁺⁾ | % H₂O | % Gas | % Residue | % loss |
|---|---|---|---|---|---|---|---|---|---|---|
| 1 | 450° | 30 | 2 | A | 0 | 15.0 | 0.5 | 83.0 | 1.5 | |
| | C. | min | | | | | | | | |
| 2 | 450° | 30 | 2 | A + 1% | 0 | 26.7 | 1.2 | 71.1 | 1.0 | |
| | C. | min | | PA | | | | | | |
| 3 | 450° | 30 | 2 | A + 5% | 0 | 23.5 | 2.3 | 73.2 | 1.0 | |
| | C. | min | | paper | | | | | | |
| Comp. 1 | 450° | 30 | 2 | A + 10% | 0 | 65.4 | 1.5 | 31.7 | 1.4 | |
| | C. | min | | PA | | | | | | |
| 4 | 450° | 30 | 3 | A | 0 | 26.0 | 2.0 | 69.0 | 3.0 | |
| | C. | min | | | | | | | | |
| 5 | 450° | 30 | 1 | A | 0 | 19.9 | 0.4 | 76.8 | 2.6 | |
| | C. | min | | | | | | | | |
| 6 | 450° | 30 | 1 | A1 | 0 | 11.7 | 0.7 | 60.3 | 28.4 | −1.1 |
| | C. | min | | | | | | | | |
| 7 | 450° | 30 | 1 | B | 0 | 20.3 | 0.9 | 82 | 0 | −3.2 |
| | C. | min | | | | | | | | |
| 8 | 450° | 30 | 1 | C | 0 | 13.2 | 0.6 | 77.4 | 12.1 | −3.3 |
| | C. | min | | | | | | | | |
| 9 | 450° | 30 | 1 | D | 0 | 18.5 | 0.8 | 81.7 | 1.9 | −2.9 |
| | C. | min | | | | | | | | |
| 10 | 450° | 30 | 1 | E | 0 | 16.6 | 0.9 | 74.4 | 7.2 | 0.9 |
| | C. | min | | | | | | | | |
| Comp. 2 | 450° | 30 | 1 | F | 0 | 36.2 | — | 52.9 | 8.9 | 1.9 |
| | C. | min | | | | | | | | |
| Comp. 3 | 450° | 30 | No | G | 0 | 57.2^{b)} | 0.7 | 31.4 | 4.4 | 6.3 |
| | C. | min | cat | | | | | | | |

^{(a)} Amount missing to 100% is due to losses;
^{b)}high amount of waxes floating in an inhomogeneous liquid
⁺⁾Liquid was the combination of the fractions, may have contained waxy solid particles and agglomerates which disappeared upon heating to >50° C. *

The above data show that the process produces high amounts of gaseous depolymerization products and maintains good performances even when the feedstock is added with heterogeneous material. The results of the analysis of the gaseous depolymerization product are summarized in Table 7.

TABLE 7
mass balance of the gaseous depolymerization product

| | Run# | | | | | | | | | | | |
|---|---|---|---|---|---|---|---|---|---|---|---|---|
| | 1 | 2 | 3 | C1 | 5 | 6 | 7 | 8 | 9 | 10 | C2 | C3 |
| | Feedstock | | | | | | | | | | | |
| | | A + 1% | A + 5% | A + 10% | | | | | | | | |
| | A | PA | paper | PA | A | A1 | B | C | D | E | F | G |
| | Composition | | | | | | | | | | | |
| | Wt. % | Wt. % | Wt. % | Wt. % | Wt. % | Wt. % | Wt. % | Wt. % | Wt. % | Wt. % | Wt. % | Wt. % |
| H₂ | 0 | 0 | 0 | 0 | 0.1 | 0 | 0.1 | 0.1 | 0.1 | 0.1 | 0.0 | 0 |
| CO | 1.8 | 2 | 3.2 | 2.7 | 0 | 0.6 | 0 | 0 | 0.0 | 0.0 | 11.3 | 4.9 |
| CO₂ | 11.2 | 11.5 | 13.9 | 11.8 | 2.4 | 3.7 | 0.6 | 0.5 | 0.5 | 2.0 | 22.3 | 18.5 |
| CH₄ | 0.5 | 0.9 | 0.7 | 5.6 | 0 | 0.5 | 0 | 0 | 0.0 | 0.6 | 5.4 | 4.3 |
| C₂H₆ | 0.4 | 1 | 0.6 | 10.8 | 0.3 | 0.4 | 0.6 | 0.7 | 0.5 | 0.4 | 7.5 | 8.8 |
| C₂H₄ | 0.9 | 1.3 | 1 | 10.2 | 0.8 | 1.1 | 1.4 | 1.5 | 2.0 | 1.6 | 6.7 | 7.8 |
| C₃H₈ | 2.1 | 2.1 | 1.9 | 11.5 | 1.3 | 3.4 | 2.7 | 3.2 | 3.4 | 0.7 | 7.0 | 9.7 |
| C₃H₆ | 27.9 | 27.7 | 25.9 | 18.9 | 33.3 | 34.6 | 28.8 | 28.3 | 29.6 | 32.1 | 14.9 | 14.3 |
| Butanes | 23.3 | 20 | 19.6 | 9.7 | 21.5 | 27.9 | 23.4 | 22.6 | 23.2 | 22.5 | 3.5 | 8.4 |
| Butenes | 32 | 33.5 | 33.2 | 18.7 | 40.4 | 27.7 | 42.4 | 43.2 | 40.7 | 40.0 | 8.9 | 11.5 |
| Rest | 0 | 0 | 0 | 0 | 0 | 0 | 0 | 0 | 0.0 | 0.0 | 12.5 | 11.9 |
| Olefins | 60.8 | 62.5 | 60.1 | 47.8 | 74.5 | 63.4 | 72.6 | 73.0 | 72.3 | 73.7 | 30.5 | 33.6 |
| total HC | 87.1 | 86.5 | 82.9 | 85.4 | 97.6 | 95.6 | 99.3 | 99.5 | 99.4 | 97.9 | 54.0 | 76.6 |
| Olefins/HC | 69.8 | 72.3 | 72.5 | 56.0 | 76.3 | 66.3 | 73.1 | 73.4 | 72.7 | 75.3 | 56.4 | 43.8 |

* Catalyst, which was used in three successive runs, maintained good selectivity and then regenerated by heating up to 800° C. in air and sieving out the smaller particles <500 μm (ashes)
HC: Hydrocarbons
Olefins: sum of ethylene, propylene and butenes
Olefins/HC: percentage of the olefins over total hydrocarbons

As can be seen from Table 7, the process yielded gaseous depolymerization products with high amounts of monomers useful as feedstock for polymerization after purification.

Comparative runs with the polymeric waste material with bulk density of 60 g/cm³ were also carried out in the same reactor set-up. The runs were impacted repeatedly by blockage of feeding line and reactor fouling which rendered the runs troublesome.

1. A process for the conversion of plastic waste into olefin, comprising the steps of:
(i) depolymerizing, at a temperature ranging from 400 to 700° C., in the presence of a catalyst, a plastic waste feedstock comprising
(a) more than 80% wt of polyolefins and
(b) less than 7.0 wt. % of non-polyolefin organic components,
based on the total weight of the dry weight polymeric fraction of the waste material feedstock, thereby generating a gaseous fraction;
(ii) collecting the gaseous fraction generated; and
(iii) separating the collected gaseous fraction, thereby obtaining a gaseous and a liquid depolymerization product comprising
higher than 60% wt of gaseous depolymerization product, based on total polyolefin content, wherein the gaseous depolymerization product comprised equal to or higher than 65% wt of C₂-C₄ olefins, based on the total amount of hydrocarbons.

2. The process of claim 1, wherein the plastic waste feedstock comprised more than 85 wt. % of polyolefin content, based on the polymeric content of the plastic waste feedstock.

3. The process according to claim 1, wherein the plastic waste feedstock (i) is in shredded form and has a bulk density from 70 to 500 g/l or (ii) is in pellet form and has a bulk density from 300 to 700 g/l, the bulk density being determined according to DIN 53466.

4. The process according to claim 1, wherein the plastic waste feedstock comprises polypropylene (PP) and polyethylene (PE), in a weight ratio PE/PP equal to or higher than 2.

5. The process according to claim 1, wherein the plastic waste feedstock further comprises less than 4% of total volatiles (TV), measured as the weight loss of a 10 g sample at 100° C. after 2 hours at 200 mbar.

6. The process according to claim 1, wherein the plastic waste feedstock comprises polymeric content consisting of polyethylene (PE).

7. The process according to claim 1, wherein the upper limit of polyolefin content is 99 wt %, based on the total amount of plastic waste feedstock.

8. The process according to claim 1, wherein the depolymerization product comprises more than 70 wt. % of gaseous content.

9. The process according to claim 8, wherein the depolymerization product comprises more than 75 wt. % of gaseous content.

10. The process according to claim 1, wherein the gaseous depolymerization product comprises equal to or higher than 70% wt of C₂-C₄ olefins, based on the total amount of hydrocarbons.

11. The process according to claim 10, wherein the gaseous depolymerization product comprises equal to or higher than 72% wt of C₂-C₄ olefins, based on the total amount of hydrocarbons.

12. The process according to claim 1, wherein the gaseous depolymerization product comprises higher than 80% of C₂-C₄ hydrocarbons, based on the total amount of hydrocarbons.

13. The process according to claim 1, wherein the catalyst comprises, as the active component, an acidic compound deposited on a particulate non-porous support with the aid of a coating agent.

14. The process according to claim 13, wherein the acidic compound is selected from the group consisting of Al/Si mixed oxides, Al₂O₃, aluminosilicates, silica, and zeolites.

15. The process according to claim 14, wherein the coating agent is selected from the group consisting of oil, inorganic hydrogel, and combinations thereof.
